(19) United States
(12) Patent Application Publication
(10) Pub. No.: US 2003/0150462 A1
(43) Pub. Date: Aug. 14, 2003

(54) Aerosol delivery apparatus-ventilator connector with adapter (VCA) and medicament dispenser and adapter (MDA)

(76) Inventors: Sunil Kumar Dhuper, Old Westbury, NY (US); Sarita Dhuper, Old Westbury, NY (US)

Correspondence Address: SUNIL DHUPER, 47, RED GROUND ROAD, OLD WESTBURY, NY, 11568, US

(21) Appl. No.: 10/099,210

(22) Filed: Feb. 14, 2002

(51) Int. Cl. A61M 16/00

(52) U.S. Cl. 128/207.14; 128/200.26; 128/202.27; 128/200.23

(57) ABSTRACT

Current methods of drug administration to the lungs are inefficient. ‘Aerosol Delivery Apparatus’ in specifically designed for uniform intrapulmonary deposition of aerosolized medication in patients on mechanical ventilation. As opposed to the current methods of drug delivery where aerosol particles are generated at the proximal end of the ET tube, with majority of the particles adhering to the endotracheal tube during delivery, this invention bypasses the endotracheal tube by generating aerosol particles at its distal end. This invention incorporates a medicament dispenser with an adapter (MDA) to perfectly fit the nozzle of a conventional metered dose inhaler and an L-shaped ventilator connector with adapter (VCA) to fit MDA. From the distal end of MDA adapter originates a semi-rigid cannula with a narrow lumen that terminates as a pinhole opening distally where aerosol particles are generated on actuation of MDI valve.

### CROSS-REFERENCE TO RELATED APPLICATIONS

| | | | |
|---|---|---|---|
| 2796295 | June 1957 | McKinnon | 239/356 |
| D184636 | March 1959 | Pickerell et al. | D9/301 |
| 2906265 | September 1959 | Samuels | D24/110 |
| 2990563 | July 1961 | Davidson | D24/119 |
| 3104062 | September 1963 | Mahon | D24/62 |
| D198964 | August 1964 | Dash et al. | D24/115 |
| 3184115 | May 1965 | Meshberg | 128/200 |
| D206979 | February 1967 | Jaffe | D24/62 |
| D207143 | March 1967 | Goodwin | D24/62 |
| 3826413 | July 1974 | Warren | 128/200 |
| D233845 | December 1974 | Fettel et al. | D24/129 |
| 4114811 | September 1978 | Loeffler | 128/400 |
| D251203 | February 1979 | Williamson | D9/352 |
| 4190046 | February 1980 | Virag | 128/200 |
| D258535 | March 1981 | Reichl | D24/62 |
| 4291688 | September 1981 | Kistler | 128/400 |
| D262320 | December 1981 | Mono | D24/62 |
| D264940 | June 1982 | Stock | D9/355 |
| D272094 | January 1984 | Wolf et al. | D24/1110 |
| 4711378 | December 1987 | Anderson | 222/206 X |
| D294175 | February 1988 | Briggs | D24/62 |
| 4830224 | May 1989 | Brison | 222/402 |
| D304232 | October 1989 | Fuller | D24/110 |
| D307183 | April 1990 | Kalayjian | D24/110 |
| D328244 | July 1992 | Hamilton et al. | D24/110 X |

### BACKGROUND OF THE INVENTION

The present invention, “Aerosol Delivery Apparatus”, relates to medical-surgical devices designed for effective intrapulmonary delivery of aerosolized medication—quantitatively as well as qualitatively (uniform distribution in tracheobronchial tree) in patients who are intubated and require mechanical ventilation. The Apparatus has two parts:

(1) Ventilator connector with adapter (VCA)

(2) Medicament Dispenser and Adapter (MDA)

The advantages of intrapulmonary drug delivery as opposed to systemic administration are well known. Many therapeutic substances can be utilized through this route, to name a few, bronchodilators, anti-inflammatory agents like steroids, antibiotics, anticholinergics, heparin, surfactant, antiproteases, gene transfer products, etc. Current methods of drug administration to the lungs are inefficient. Not only are they limited in delivery of quantitatively significant amount of medication to the lungs, but they have also failed qualitatively to achieve uniform intrapulmonary distribution.

There are two methods currently available for intrapulmonary drug delivery.

(I) Liquid bolus: The medication is instilled in the form of liquid bolus via a bronchoscope or through an endotracheal (ET) tube. Not only is the distribution by this method non-uniform but there is also a significant risk of inducing respiratory distress and hypoxemia.

(II) Aerosol Inhalation: Methods employed use Metered Dose Inhalers (MDI's) with low boiling point propellants such as chlorofluoroalkanes or aerosol particles generated by heat, traditional compressed air nebulizers, or ultrasonic nebulizers. This method, even though it produces a more uniform distribution of aerosol particles compared with liquid bolus method, is limited in quantitatively delivering significant amount of medication to the lungs. Only a small fraction of the medication reaches the lungs and majority of the aerosol particles either adhere to the nasal passages and oropharynx or are exhaled out. Efficiency of aerosol delivery drops down even further in patients who are intubated and on mechanical ventilation. Beck et al found that inhalation of nebulized material through an endotracheal tube resulted in deposition of only 1.87% of the delivered particles to the lungs.

Investigators over the years have devised numerous endotracheal tubes for intrapulmonary drug delivery. Most designs of endotracheal tubes so far have only addressed the issue of drug delivery in the form of liquid bolus by incorporating drug irrigation devices in the traditional ET tube either in the form of secondary canalization with multiple micrometric openings (U.S. Pat. No. 5,146,936) or with some such modification of the original design.

Generation and delivery of aerosol particles with small mid-mean diameter, which is critical for uniform deposition in the tracheobronchial tree especially to reach the small airways, has not been addressed by any of the currently existing endotracheal tubes incorporating drug irrigation devices. Recently one of the investigators invented a delivery device for intratracheal administration of drug in aerosol form called ‘Penn Century Intratracheal Aerosolizer (Microsprayer)’ [U.S. Pat. Nos. 5,579,758, 5,594,987, 5,606,789, 5,513,630, 5,542,412, 5,570,686]. This device is not related to our field of invention. The clinical utility of this device in humans at this time is extremely limited because of its high cost and need for sterilization after every use and as such it is solely being used as a research tool.

### BRIEF SUMMARY OF THE INVENTION

Objects of Invention

The main object of the present invention is to provide a new aerosol delivery apparatus that serves the following purposes:

Aerosol drug delivery to tracheobronchial tree.

Generation and delivery of aerosol particles at the distal end of the ET tube with mid mean diameter that will allow uniform distribution throughout the tracheobronchial tree.

Generation and of delivery of aerosol particles at the distal end of the ET tube so as to quantitatively deliver significant fraction of the generated aerosol particles to the tracheobronchial tree without adherence to the ET tube. This also implies cost effectiveness by preventing waste of medication.

Simple inexpensive method of intrapulmonary drug delivery

To achieve all the previous mentioned objects without interfering with the function of the ET tube.

To achieve the above objectives through a device that does not make it more complicated for the operator to deliver the medication or is in anyway traumatic to the patient.

The defined objects are obtained through our invention that incorporates the following new features:

Ventilator Connector with Adapter: (VCA): The Ventilator connector has been specially designed to be L-shaped. VCA should be placed in the circuit between the ET tube and ventilator tubing only as “inverse L” (┌). The vertical part of VCA is connected to the ET tube through a connector and the horizontal part of VCA is connected to the ventilator. On the horizontal limb of the VCA more towards the junction where horizontal and vertical limbs meet, there is an adapter for accommodating MDA (described below). The purpose of “┌” is to ensure that the lumen of the adapter on the horizontal surface of the VCA is in straight line with the lumen of vertical limb; in other words in alignment with the ET tube. Any other position of the adapter would not allow for the semi-rigid cannula of MDA to be manipulated through the lumen of the ET tube. A flexible cannula would completely defeat the purpose of our invention as the pressure generated by actuating the MDI valve would result in a fling in the catheter and fail to generate aerosol particles at its tip. The cylindrical adapter on the horizontal limb of the VCA is designed such that its inner circumference forms a perfect framework to fit the framework of the outer circumference of the cylindrical MDA. When MDA and VCA are not in connection, the adapter of VCA remains closed with a rubber plug or cap making an air tight seal.

Medicament Dispenser and Adapter (MDA)—This equipment is specifically designed such that the outer circumference of the cylindrical nozzle located at the end of a conventional Metered Dose Inhaler (MDI) cannister perfectly fits into the inner circumference of the proximal cylindrical framework of MDA. Aerosol particles are generated when the MDI valve is actuated. MDA is also designed such that the outer circumference of its cylindrical framework perfectly fits into the inner circumference of the cylindrical framework of VCA adapter (described above), located on the horizontal limb of VCA. The MDA tapers near its distal tip reach an inner diameter (ID) of a pinhole. The distal tip of the adapter marks the origin of a semi-rigid cannula that continues distally for a variable length to terminate as a pinhole opening. This feature of our invention differentiates it from most of the other existing adapters that have a pinhole opening on one of the sides, a few millimeters higher than the distal end of the adapter which is generally closed. The length of the cannula can vary depending on the size of the ET tube (approximately 38 cm for an adult size ET tube). The rigidity of the cannula could be altered by increasing the thickness of its wall and using a stiffer plastic material. The semi-rigid cannula with a narrow lumen ensures that the particles generated by MDI reach its distal tip in aerosol form without producing any fling in the cannula. The use of an MDI for intrapulmonary delivery of various medications is well known. An MDI consists of a pressurized cannister containing powdered medication with a low boiling point propellant maintained in liquid state. When the valve of the MDI is activated, the propellant is released and forces medicament from the nozzle of the cannister along with propellant. Since the essence of this invention disclosed herein does not relate specifically to the structure of an MDI device, the details of this construction will not be discussed herein. Means of making and using MDI are well known to those skilled in the art.

MDA & VCA in Connection: When the two parts are in connection, the cannula of the MDA extends all the way through the lumen of the ET tube to end just distal to the tip of the ET tube. The MDI at the proximal end of MDA generates particles that are delivered in aerosolized form beyond the tip of the ET tube to the tracheobronchial tree.

### DESCRIPTION OF THE SEVERAL VIEWS OF THE DRAWING

Further features of the present invention will become apparent in the accompanying drawings as well as the detailed description of the preferred embodiments.

FIGS. 1a, 1b, & 1c show the perspective views of the ventilator connector with adapter (VCA).

FIG. 1a represents the oblique view of the VCA with a plug or cap.

FIG. 1b represents the cross section view (top and bottom views) of the horizontal limb of VCA.

FIG. 1c represents the cross section view (top and bottom views) of the vertical limb of VCA.

FIGS. 2a, 2b and 2c show the perspective views of medicament dispenser and adapter (MDA).

FIG. 2a represents the oblique view of the MDA

FIG. 2b. represents the front, rear, left and right elevational views of the MDA.

FIG. 2c. represents the cross section view (top and bottom views) of the MDA.

FIG. 3 represents VCA and MDA in connection.

### DETAILED DESCRIPTION OF THE INVENTION

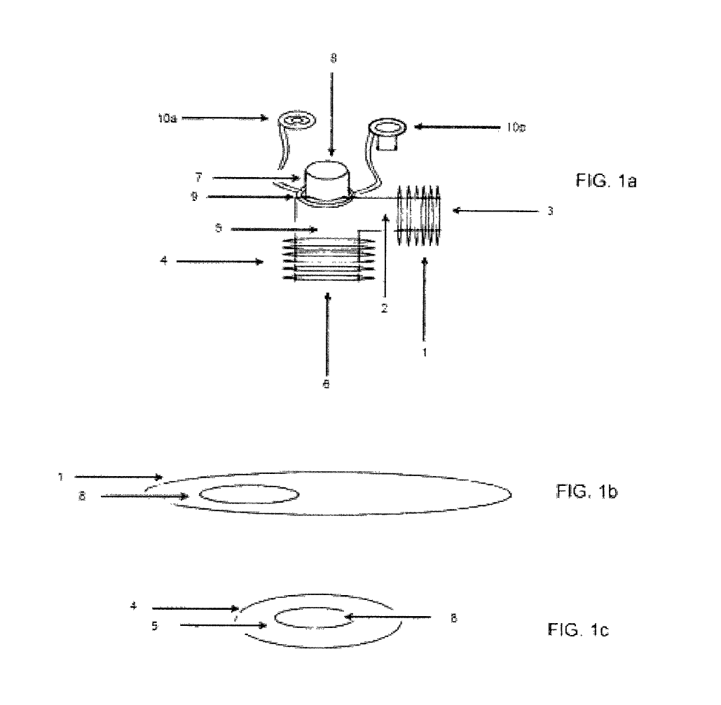

FIGS. 1a, 1b & 1c outline the design of VCA.

1a represents the oblique view of the inverse L shape VCA—“┌”.

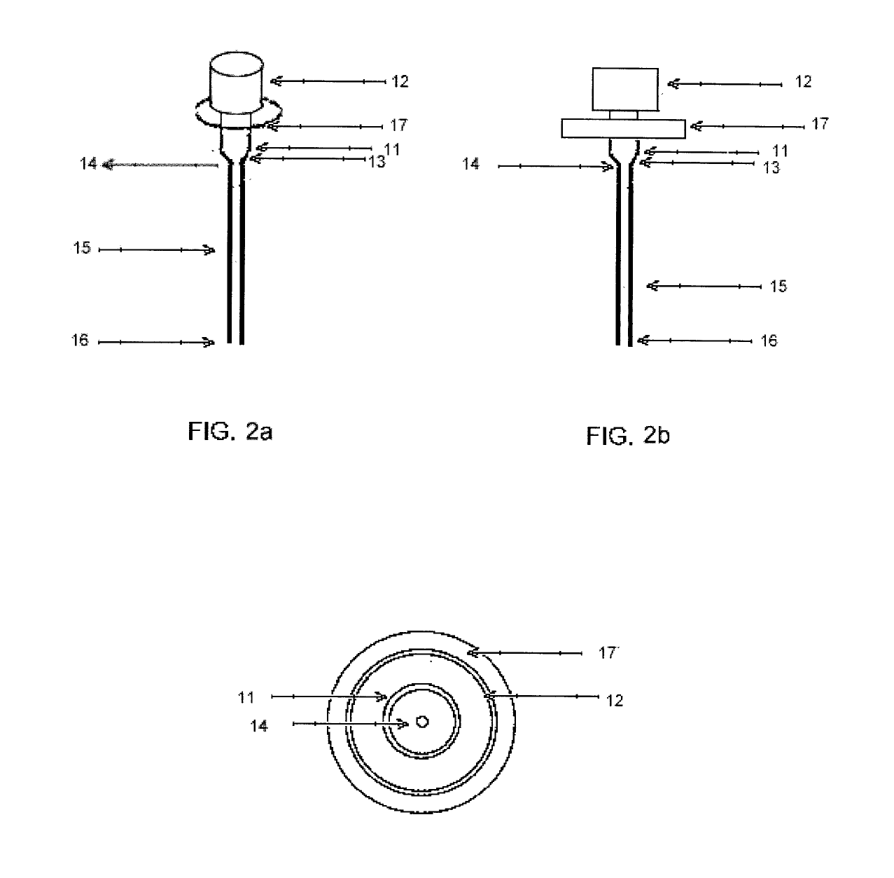

“┌” has a horizontal limb (1) with a lumen (2) and a terminal end (3) for connection to corrugated tube for the ventilator. “┌” has a vertical limb (4) with a lumen (5) and a terminal end (6) for connection to the ET tube. There is an adapter (7) on the horizontal limb of “┌” with a lumen (8). The inner circumference of the cylindrical adapter (7) is designed such that the outer circumference of the distal cylindrical MDA (11) (described in FIG. 2) tightly fits into it. There is a plastic or rubber ring around the adapter (9) with a cap (10a) or a plug (10b) that locks into the adapter to make an air tight seal when MDA is not in connection with VCA. The purpose of “┌” is to ensure that the adapter lumen (8) its in straight line with the lumen (5) of the vertical limb (4). This will ensure that the semi-rigid cannula of MDA (18) can be easily manipulated through the lumen of the ET tube to terminate distally beyond the tip of the ET tube.

1b, that represents the cross section view of the horizontal limb of VCA (top and bottom views), demonstrates the horizontal limb (1) and adapter lumen (8).

1c, that represents the cross section view of the vertical limb of VCA (top and bottom views), demonstrates the vertical limb (4), its lumen (5) and the adapter lumen (8).

FIGS. 2a, 2b and 2c outline the design of MDA.

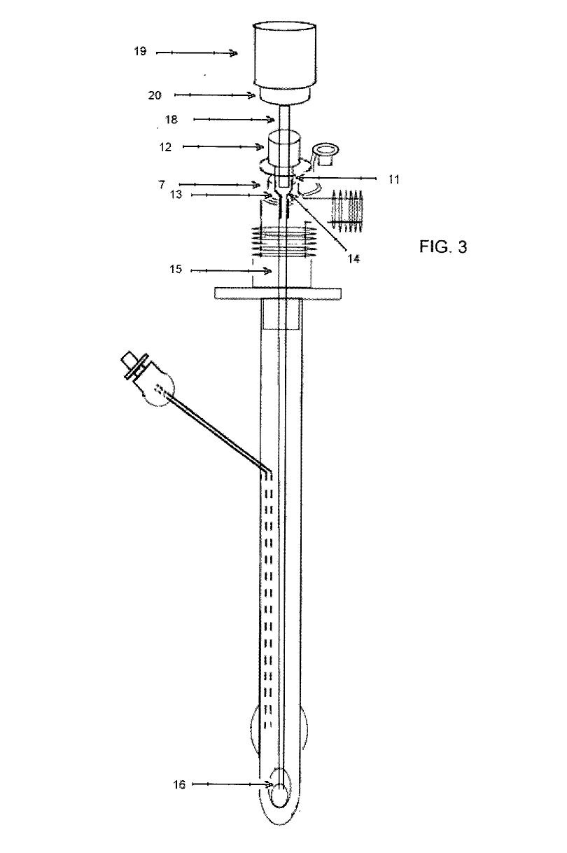

FIG. 2a, the oblique view of MDA, is in the shape of a cylinder—the proximal cylinder (12) with a greater circumference than the distal cylinder (11). The inner circumference of distal cylinder (11) is designed to fit outer circumference of the cylindrical nozzle (18) of the MDI (19) (described in FIG. 3). The outer circumference of distal cylinder (11) is designed to fit the inner circumference of VCA adapter (7). The distal cylindrical adapter (11) continues distally for 1-2 mm and tapers at the tip (13) to reach an ID of a pinhole (14) which marks the origin of a semi-rigid cannula (15). The semi-rigid cannula (15) continues distally for a variable length to terminate as a pinhole opening (16). The ID of the cannula and pinhole tip could vary from 0.15 mm to 2.0 mm. The length of the cannula could vary depending on the size of the ET tube such that it terminates approximately ½ cm distal to the tip of the ET tube (approximately 38 cm for a full adult size ET tube). The rigidity of the cannula could be modified by changing the thickness of the wall and using a variety of plastic materials in order to allow manipulation within the lumen of the ET tube avoiding the fling phenomenon. Wrapped around the center of MDA is a plate like circular frame (19). This is an optional feature of our invention. This circular frame rests on top of the VCA adapter (7) when the distal cylindrical frame of MDA (11) fits into it. The circular plate is an additional safety measure to ensure that MDA does not slip into the inspiratory circuit.

FIG. 2b represents the four perspective views of MDA—the front, rear, right and left as described in FIG. 2a

FIG. 2c represents the cross section view of MDA (top and bottom views) as described in FIG. 2a. The proximal cylinder (12) the distal cylinder (11), the terminal opening (14) and an optional circular plate (17) are described here.

FIG. 3 represents the perspective view of MDA and VCA in connection. VCA and MDA stay disconnected at all times unless the delivery of medication is required. The VCA adapter remains plugged (sealed airtight) at all times. When ready to deliver the medication, the VCA adapter is unplugged and the semi rigid cannula (15) is manipulated distally all the way through the lumen of the ET tube to reach its tip. Finally at this point, a connection is made between the distal cylindrical part of MDA (11) and VCA adapter (7). The nozzle (18) of the MDI cannister (19) can now be plugged into MDA adapter (11), the MDI valve is actuated by pressing the MDI (20) with thumb and aerosol particles are generated and delivered to the terminal end of the cannula (16). MDA and VCA should be disconnected immediately after delivery of medication and VCA adapter re-plugged. In case VCA and MDA are not disconnected, there may be an increase in the airway pressure (increased resistance) in the circuit due to reduced radius of the lumen.

It is noted that the illustration (drawings) and description of the preferred embodiments have been provided merely for the purpose of explanation and although the invention has been described herein with reference to particular means, materials and embodiments, the invention is not intended to be limited to the particular disclosed herein; rather the invention intends to all functionally equivalent structures, methods and uses such as are within the scope of the appended claims.

We claim:

1. an Aerosol Delivery Apparatus
with two parts—VCA and MDA
with capability of quantitative and qualitative (uniform) delivery of aerosolized particles of medication to the tracheobronchial tree in intubated patients.

2. A VCA
which is “L” shaped
which should be used in the circuit only as inverse L—“┌”.
with a provision for an adapter on the horizontal limb of the “┌”.
with an adapter on the horizontal limb such that its lumen is in straight line with the lumen of the vertical limb of “┌”.
with an adapter whose framework perfectly fits the framework of MDA

3. An MDA
with provision for aerosol delivery of medications to the lungs via a metered dose inhaler (MDI)
with an adapter designed to fit the nozzle of MDI cannister.
with cylindrical shape such that fits into the cylindrical framework of the VCA adapter.
which tapers at its distal tip to reach an inner diameter (ID) of an pinhole that marks the origin of a semi-rigid cannula.
with a semi-rigid cannula that could be manipulated distally through the lumen of the ET tube
with a cannula without fling when the pressure valve of MDI is actuated
with a cannula whose ID is small enough for the particles generated by a MDI at the proximal end to be delivered at the distal tip of the ET tube in aerosol form
with a proximal cylindrical part whose circumference is greater than the outer circumference of the VCA adapter to prevent MDA from slipping into the inspiratory circuit
with an additional safety feature in the form of a circular plate wrapped around the MDA to prevent it from slipping into the vertical limb of VCA.
